(19) United States
(12) Patent Application Publication
(10) Pub. No.: US 2024/0108413 A1
(43) Pub. Date: Apr. 4, 2024

(54) PHYSICAL IOL POSITION DETERMINATION ON THE BASIS OF DIFFERENT INTRAOCULAR LENS TYPES

(71) Applicant: Carl Zeiss Meditec AG, Jena (DE)

(72) Inventors: Hendrik Burwinkel, Oberkochen (DE); Michael Trost, Oberkochen (DE); Nicolas Bensaid, Oberkochen (DE); Stefan Saur, Oberkochen (DE)

(73) Assignee: Carl Zeiss Meditec AG, Jena (DE)

(21) Appl. No.: 18/476,245

(22) Filed: Sep. 27, 2023

(30) Foreign Application Priority Data
Sep. 30, 2022 (DE) 102022125421.6

(51) Int. Cl. A61B 34/10

(52) U.S. Cl. CPC A61B 34/10; A61B 2034/104; A61B 2034/105; A61B 2034/107; A61B 2034/108

(57) ABSTRACT

A computer-implemented method for training a machine learning system to determine an expected offset for a physical postoperative lens position of an intraocular lens to be inserted. The method includes determining a plurality of theoretical positions in the eye of different intraocular lenses to be inserted, the determination including a respective use of a relation and a respective lens-specific constant for the plurality of the theoretical postoperative positions. The method includes: measuring a plurality of postoperative positions of the intraocular lens, with the real postoperative positions being assigned to respective associated theoretical postoperative positions; determining positional differences between theoretical postoperative positions and real postoperative positions; measuring associated ophthalmological biometry data for each tuple; and training a machine learning system to form a trained machine learning model for a prediction of the expected offset for a physical postoperative lens position.

### CROSS REFERENCE TO RELATED APPLICATIONS

This U.S. patent application is a continuation of, and claims priority under 35 U.S.C. § 119(d) to German Patent Application DE 10 2022 125 421.6, filed on Sep. 30, 2022. The disclosure of this prior application is considered part of the disclosure of this application and is hereby incorporated by reference in its entirety.

### TECHNICAL FIELD

This disclosure relates to a method for training a machine learning system and to a method for determining an expected offset for a physical postoperative lens position of an intraocular lens to be inserted. The disclosure also relates to a training system for training a machine learning system and to a prediction system for determining a real postoperative position of an intraocular lens using the machine learning system, and to a computer program product.

### BACKGROUND

These days, ever more scientific fields are stimulated by the use of artificial intelligence (AI). AI and machine learning (ML) systems, which use prediction models, have also found use in the daily routine of medical practitioners, for example within the scope of cataract operations. The natural eye lenses are replaced by artificial lenses—so-called intraocular lenses—during these operations. Determining a refractive power of the intraocular lens to be inserted prior to an operation is very important to the surgeon since an incorrectly chosen intraocular lens cannot be adjusted retrospectively. Machine learning systems are already used successfully for a determination of a postoperative position of the inserted intraocular lens, and consequently also for a determination of the resultant refractive power. To allow these learning systems to make predictions that are as reliable as possible, it is necessary for them to be trained using a sufficient amount of training data such that a machine learning model which enables reliable predictions arises.

However, on the downside, there are a multiplicity of different types of intraocular lenses. Even if the nominal refractive power of two types of intraocular lenses is identical, there may be a difference in the effective postoperative refractive power that is developed within the patient's eye by the inserted intraocular lens. Among other things, this may be due to the fact that the inserted intraocular lens, depending on its type, grows into a different plane of the patient's eye.

These circumstances lead to the fact that data collected from cataract operations contain different amounts of data in relation to different intraocular lens types. Naturally, it is also recurrently the case that only a small amount of training data is available for certain lens types and refractive powers. Since this absolutely required amount of training data may under certain circumstances be too small—or undershoots a critical amount—to train an effective learning model, it may be the case that a surgeon may not be able to resort, or may only be able to unreliably resort, to the aid of machine learning systems in such cases.

In view of this situation, there therefore is the desire in cataract surgery of resorting to the assistance of machine learning systems even if only a small amount of training data is available, in order to thus obtain the greatest possible success during operations. It would be ideal if training data originating from different types of intraocular lenses could be used to develop the machine learning model. However, the different properties of the different intraocular lens types make the learning process of the machine learning system more difficult as a result of a domain-related shift of the different lens types, and this may lead to a poorer prediction quality of the trained machine learning system if this problem is not addressed accordingly.

Consequently, there is a need to be able to train high-performance machine learning systems, even if only a small amount of training data is available for individual lens types.

### SUMMARY

One aspect of the disclosure provides a computer-implemented method for training a machine learning system to determine an expected offset for a physical postoperative lens position of an intraocular lens to be inserted is presented. In the process, the method includes determining a plurality of theoretical postoperative positions in the eye of intraocular lenses to be inserted, the intraocular lenses to be inserted belonging to different types of intraocular lenses and the determination including a respective use of a relation and a respective lens-specific constant for the plurality of the theoretical postoperative positions.

Furthermore, the method may include measuring a plurality of real postoperative positions—i.e., real positions after an operation—of the intraocular lens, with the real postoperative positions being assigned to respective associated theoretical postoperative positions and forming a tuple in each case.

Additionally, the method may include determining positional differences between theoretical postoperative positions and real postoperative positions which are respectively associated with one another, and measuring associated ophthalmological biometry data for each tuple.

Finally, the method may also include training a machine learning system to form a trained machine learning model for a prediction of the expected offset for a physical postoperative lens position for a specific type of intraocular lens to be inserted, with the ophthalmological patient data for each tuple and associated positional differences being used as input data for training the machine learning system.

According to a second aspect of the disclosure, a method for determining a real postoperative position of an intraocular lens using the machine learning system is presented, the latter having been trained by means of the aforementioned method. In this case, the method includes: using the relation for determining the theoretical postoperative lens position of the intraocular lens to be inserted, determining ophthalmological biometry data from a measurement on the patient's eye, determining, by means of the trained machine learning system, the expected offset for the physical postoperative lens position for an intraocular lens to be inserted, with the determined ophthalmological biometry data being used as input data, and determining a corrected postoperative lens position by adding the theoretical postoperative lens position of the intraocular lens to be inserted and the determined expected offset for the physical postoperative lens position.

According to a third aspect of the disclosure, a method for determining a postoperative refractive result is presented, which includes determining the real postoperative position of an intraocular lens using the above-described method.

This method additionally includes determining the expected postoperative refractive result of a cataract surgery by means of a trained machine learning system, with a real refractive power value of the intraocular lens to be inserted and the determined real postoperative position of an intraocular lens being used as input data, and the machine learning system having been trained with tuples of postoperative lens position values, real refractive power values of corresponding intraocular lenses, and associated postoperative refractive results as ground truth data, in order to form a corresponding learning model for predicting the postoperative refractive result of the intraocular lens to be inserted.

According to a fourth aspect of the disclosure, a training system for training a machine learning system to determine an expected offset for a physical postoperative lens position of an intraocular lens to be inserted is presented. This training system comprises a processor and a memory which is operatively connected to the processor and which stores program code elements which, when executed, are active for a cooperation of the following units:
a determination module for determining a plurality of theoretical postoperative positions in the eye of intraocular lenses to be inserted, the intraocular lenses to be inserted belonging to different types of intraocular lenses, the determination including a respective use of a relation and a respective lens-specific constant for the plurality of the theoretical postoperative positions;
a first measurement system for measuring a plurality of real postoperative positions of the intraocular lens, with the real postoperative positions being assigned to respective associated theoretical postoperative positions and forming a tuple in each case;
a determination unit for determining positional differences between theoretical postoperative positions and real postoperative positions which are associated with one another in each case,
a second measurement system for measuring associated ophthalmological biometry data for each tuple; and a training control unit for training a machine learning system to form a trained machine learning model for a prediction of the expected offset for a physical postoperative lens position for a specific type of intraocular lens to be inserted, with the ophthalmological patient data for each tuple and associated positional differences being used as input data for training the machine learning system.

According to a fifth aspect of the disclosure, a prediction system for determining a real postoperative position of an intraocular lens using the machine learning system is presented, the latter having been trained by means of the training system described just hereinabove.

In this case, the prediction system comprises a processor and a memory which is operatively connected to the processor and which stores program code elements which, when executed, are active for a cooperation of the following units:
a relation determination unit for using the relation for determining the theoretical postoperative lens position of the intraocular lens to be inserted;
an eye data determination unit for determining ophthalmological biometry data from a measurement on the patient's eye;
an offset determination unit for determining, by means of the trained machine learning system, the expected offset for the physical postoperative lens position for an intraocular lens to be inserted, with the determined ophthalmological biometry data being used as input data, and
a lens position unit for determining a corrected postoperative lens position by adding the theoretical postoperative lens position of the intraocular lens to be inserted and the determined expected offset for the physical postoperative lens position.

The proposed computer-implemented method for training a machine learning system for determining an expected offset for a physical postoperative lens position of an intraocular lens to be inserted and the further methods have a plurality of advantages and technical effects, which also apply accordingly to the respectively associated systems:

Among other things, the proposed methods and systems advantageously address the problem of training a machine learning system such that a high performance machine learning model for predicting a postoperative position of an intraocular lens in the eye of a patient arises even if only a comparatively small amount of training data is available for individual types of intraocular lenses. Conversely, this means that the data consisting of a mixture of specifications in relation to different types of intraocular lenses can be used as training data.

This is also advantageous because a machine learning system can be used to make a reliable prediction of the postoperative position of an intraocular lens on the basis of the type of intraocular lens. That is to say there is no need to train and operate a dedicated machine learning system for each type of intraocular lens. That is to say even an offset between different types of intraocular lenses in relation to the postoperative position thereof in the eye can be reliably predicted by means of the trained machine learning system. A reliable prediction in relation to the real postoperative refractive power perceived by the patient can also be made on this basis. This even applies if only a small amount of training data was available at the outset for the training of the machine learning system for developing the underlying learning model.

To this end, a theoretical lens position calculation and a known value for a specific lens type or a specific lens are drawn upon. What this achieves is that the critical distance of the lens from the cornea can be included in the prediction of the postoperative refractive power as this distance influences the real postoperative refractive power.

Previous approaches in relation to theoretical effective positioning of the intraocular lens within an individual modeling concept—and in particular differences between different intraocular lens types—could not successfully address this problem to date.

Let Para_IOL be parameters containing lens-specific information and Para Patient be parameters containing patient-specific information (typically biometry data). Even though the simplest approach would be that of integrating the parameters Para_IOL and Para Patient into the training process, this would not be a practicable way that reliably leads to the sought-after target. This is due to the fact that the parameters Para_IOL in particular are typically determined by constants, for example the known A-constant for the known SRK/T formula. This leads to the system only learning isolated data points for different, isolated types of intraocular lenses during the training of the machine learning system. Even a small modification in the corresponding constant would lead to a completely unexpected behavior of the machine learning system because the machine learning system would not have “seen” comparable input data during the training.

Hence, a different approach is chosen here, based on a relation ELP=R(Para_IOL). In this case, ELP stands for an effective postoperative lens position; R stands for a relation; Para_IOL stands for the lens-specific parameters for the type of intraocular lens, for example the A-constant, used in the relation.

Now let us consider different data inputs for two different types of intraocular lenses Para_IOL1 and Para_IOL2. It emerges that the difference between effective IOL positions can be represented as ΔELP=R(Para_IOL1)−R(Para_IOL2).

Even though the effective lens position (ELP) and the physical lens position (PLP) do not depend directly on one another, they nevertheless each define an optical plane within the system of the eye. It follows from this that the shifts of the effective lens position and physical lens position, and hence the shifts of the optical planes connected therewith, correspond to one another to a first approximation, with the result that it is possible to deduce that the following applies approximately: ΔELP=ΔPLP.

The following emerges directly therefrom: ΔPLP=R(Para_IOL1)−R(Para_IOL2).

Using this, it is possible to achieve the goal of compensating all differences between different data domains which belong to different types of intraocular lenses—i.e., IOL1 vs. IOL2.

This in turn allows the surgeon to optimize the ML-based calculation model for a first type of intraocular lens IOL1 using data for another type of intraocular lens IOL2. For this reason, the PLP value can advantageously be integrated into the training process for the machine learning model. On the basis of measured postoperative IOL positions PLP for a given set of training data, a PLP prediction system is represented by:

PLP=R(Para_IOL)−Correction system (Para Patient) (1)

In this case, the correction system adopts the role of correcting a deviation vis-à-vis the relation R in relation to a current lens position, which in turn can be represented for example by an ML model or comparatively simple linear regression. The presented approach separates the lens-specific and patient-specific parameters from one another. The relation R supplies the theoretical lens position according to Para_IOL, and the correction system learns the required offset for the prediction of the actual PLP depending on Para Patient. Because the value PLP consequently is a uniformly distributed variable, the problem of only isolated data points being considered in the machine learning system is avoided. Moreover, it follows from equation (1) specified above that it is possible to derive two properties which directly help in addressing the domain shift problem (caused by the different types of intraocular lenses):

Firstly: If, after the surgical procedure, corresponding IOL positions PLP are collected for all types of intraocular lenses present in the training data record, then the PLP prediction system can operate on the basis of equation (1) described above. On account of the relation R, which eliminates the systematic differences between different types of intraocular lenses in the PLP IOL position, the corresponding machine learning system (correction system) can predict correct and identical correlations for all types of intraocular lenses. The resultant PLP-predicting machine learning system can consequently advantageously also be used for all types of intraocular lenses.

On the basis thereof, the PLP (physical postoperative lens position) determined thus can therefore be used as an input value for a machine learning system which is used for a refractive power determination of an intraocular lens to be inserted. Thus, the presented correction system can now be used to provide the physical postoperative lens position PLP for new data points and used as input values for the prediction of the refractive power of the intraocular lens to be inserted.

Secondly: Even if the postoperative IOL positions of only a few types of intraocular lenses within the training data record are known, it is nevertheless possible to realize a prediction system for the aforementioned equation (1) on the basis of the available PLP values. A transfer to other types of intraocular lenses, without the corresponding machine learning system having been explicitly trained to this end, may be successful on account of the relation R.

As a result, the use of PLP within the machine learning model enables a continuous and hence generally usable option for training with training data belonging to different types of intraocular lenses.

As a result, it is also possible to successfully address the following fields of application in daily medical routine—for example, in the case of cataract operations:
(i) collecting data for a type of intraocular lens, for which a machine learning system for a prediction of a refractive power of an intraocular lens is intended to be optimized;
(ii) making available additional data on the basis of other types of intraocular lenses;
(iii) training a correction function using all available data for all intraocular lenses and providing the corresponding ground truth data;
(iv) using the correction function within the predictions of PLP values;
(v) training the machine learning model and using the prediction/correction function and collecting data belonging to different types of intraocular lenses (in this case, the focus should be placed on desired types of intraocular lenses and this should take account of the fact that different weightings in the loss function are used during the ML system training, a repetition of the training with desired data points, . . . );
(vi) using the resultant machine learning system for determining an IOL refractive power of a specific type of intraocular lens for new patients.

The last point in particular is very important from the point of view of patients. This is because the work of surgeons within the scope of cataract operations can be improved significantly and the risk of the inserted intraocular lens growing into a different plane of the eye than planned can also be significantly reduced even if there were only a few data points for training of the used ML system as would be required by the specific situation of the current patient.

The methods and systems proposed here thus address a broad spectrum of advantages over the previous procedure, especially if only small amounts of data are available for the training of a machine learning system for specific types of intraocular lenses. A specific example will still be given herein below.

Further exemplary embodiments are presented below, which can have validity both in association with the presented methods and in association with the corresponding systems.

According to an exemplary embodiment of the computer-implemented method for training a machine learning system to determine an expected offset for a physical postoperative lens position of an intraocular lens to be inserted, the relation can be based on an SRK/T relation. The SRK/T-R relation and the associated SRK/T formula has been used in ophthalmology for quite some time and can be traced back to the authors Sanders, Retzlaff and Kraff.

According to another exemplary embodiment of the computer-implemented method, the ophthalmological biometry data of a patient may include at least one measurement value of an eye selected from the group consisting of a current axial length (AL), an anterior chamber depth (ACD), and a lens thickness. However, all three biometric data of an eye are typically acquired simultaneously using known measurement instruments.

According to further exemplary embodiments of the computer-implemented method, the machine learning system can include a linear regression model. This is advantageous from the point of view of thus ensuring that even those regions for which no explicit training data were available are captured. Hence there can even be meaningful predictions for those regions where there are no historic training data for the same type of intraocular lens or for where the desired prediction region is located between known types of intraocular lenses, which is to say even for novel intraocular lens types with other A-constants.

According to specific exemplary embodiments of the computer-implemented method, the prediction of the expected offset for the physical postoperative lens position for the specific type of intraocular lens to be inserted can be independent of a refractive power of the intraocular lens to be inserted. A clear advantage can also be seen herein since it is possible to make do without knowledge of the refractive power when using the relation in the calculation of the theoretical position of the intraocular lens post-surgery.

According to an advantageous exemplary embodiment of the computer-implemented method, the lens-specific constant can be the A-constant. This constant is well known for every type of intraocular lens and essentially describes the theoretical position of the IOL within the SRK/T relation.

Express reference is made to the fact that practically all methods and systems can be modified by corresponding versions of the aforementioned exemplary embodiments.

Furthermore, practically all embodiments may relate to computer program products able to be accessed from a computer-usable or computer-readable medium that comprises program code for use by, or in conjunction with, a computer or other instruction processing systems. In the context of this description, a computer-usable or computer-readable medium can be any device that is suitable for storing, communicating, transferring, or transporting the program code.

### DESCRIPTION OF DRAWINGS

It is pointed out that exemplary embodiments of the disclosure may be described with reference to different implementation categories. In particular, some exemplary embodiments are described with reference to methods, whereas other exemplary embodiments may be described in the context of corresponding devices. Regardless of this, it is possible for a person skilled in the art to identify and to combine possible combinations of the features of the method and also possible combinations of features with the corresponding system from the description above and below—if not specified otherwise—even if these belong to different claim categories.

Aspects already described above and additional aspects of the present disclosure will become apparent inter alia from the exemplary embodiments described and from the additional further specific configurations described with reference to the figures.

FIG. 1 illustrates a flowchart-like representation of an exemplary embodiment of the computer-implemented method according to the disclosure for training a machine learning system to determine an expected offset for a physical postoperative lens position of an intraocular lens to be inserted.

FIG. 2 shows an eye with potential parameter measurement values.

FIG. 3 shows a flowchart-like representation of an exemplary embodiment for determining a corrected post-operative lens position using the method according to FIG. 1 as a starting point.

FIG. 4 shows a flowchart-like representation of an exemplary embodiment for determining an expected postoperative refractive result of a cataract operation.

FIG. 5 illustrates a schematic representation for training a machine learning system in accordance with an exemplary embodiment of the presented concept.

FIG. 6 illustrates a schematic representation for a PLP prediction on the basis of a machine learning system which was trained by means of the training according to FIG. 5.

FIG. 7 shows a schematic representation of a training system for training a machine learning system to determine an expected offset for a physical postoperative lens position of an intraocular lens to be inserted.

FIG. 8 shows a schematic representation for determining a postoperative real position of an intraocular lens using the machine learning system that was trained by means of the training system.

FIG. 9 shows a schematic representation of a computer system which can be used in the context of the systems described herein.

Like reference symbols in the various drawings indicate like elements.

### DETAILED DESCRIPTION

In the context of this description, conventions, terms and/or expressions should be understood as follows:

The term “machine learning system” describes a system which generates prediction values (predictions) on the basis of input data. In this case, a machine learning system has not been programmed procedurally but “learns” its behavior on the basis of training with training data. In so doing, the machine learning system is provided with input data and desired output data as so-called “ground truth data”. Machine learning systems for the context specified here are typically based on neural networks. These can be implemented both as a software construct and in hardware. In addition to prediction data, machine learning systems are also able to provide an associated quality statement, for example in the form of “the predicted value is correct with a probability of x %”.

In this context, the difference between a machine learning system and a machine learning model should also be highlighted. The machine learning system represents the fundamental architecture of the learning system while the machine learning model is formed during the course of the training with training data.

The term “expected offset for a physical postoperative lens position” may describe a difference between a theoretically calculated postoperative lens position and the actually realized lens position after the operation.

The term “intraocular lens” describes an artificial lens in this case, the latter being inserted into an eye by a surgeon, to replace the natural lens of an eye. A cataract operation is a typical application.

The term “theoretical postoperative position” describes the position—i.e., the optical plane in which an intraocular lens inserted into a patient's eye is located on the basis of a relation—which is used in a corresponding calculation model.

The term “types of intraocular lenses” describes different types of intraocular lenses. By way of example, these may differ in terms of the material, the shape or the anchoring side arms around the actual lens.

In this context, the term “lens-specific constant” describes constants such as the so-called A-constant, which is frequently used to calculate lens position or refractive power.

The term “relation” describes a mathematical relationship which uses parameters as arguments of a function in order to calculate an output value.

The term “real postoperative position”—especially of an intraocular lens—does not describe a calculated or predicted position of the intraocular lens but the measured position of the intraocular lens or of a corresponding plane after the lens has grown-in in the eye.

The term “positional difference” can describe the real postoperative position of an intraocular lens in comparison with a prediction by an ML system or by a theoretical formula.

In the context of this disclosure, the term “ophthalmological biometry data” may predominantly describe the following variables of an eye: axial length (AL), anterior chamber depth (ACD) and the lens thickness of an eye.

The term “SRK/T-R relation”—or modified or related formulas—describes the known mathematical function for calculating a theoretical postoperative lens position within the SRK/T formula.

The term “SRK/T formula”—or modified or related formulas—describes the known mathematical function for calculating a theoretical postoperative refractive power of an intraocular lens on the basis of the assumed position of the intraocular lens.

The term “linear regression model” describes the known statistical procedure, within the scope of which attempts are made to explain an observed dependent variable by way of one or more independent variables. In this case, a linear model is assumed within the scope of the linear regression.

A detailed description of the figures is given below. It is understood in this case that all of the details and information in the figures are illustrated schematically. What is illustrated first is a flowchart-like representation of an exemplary embodiment of the computer-implemented method according to the disclosure for training a machine learning system to determine an expected offset for a physical postoperative lens position of an intraocular lens to be inserted. Further methods and exemplary embodiments or exemplary embodiments for the corresponding systems are described herein below:

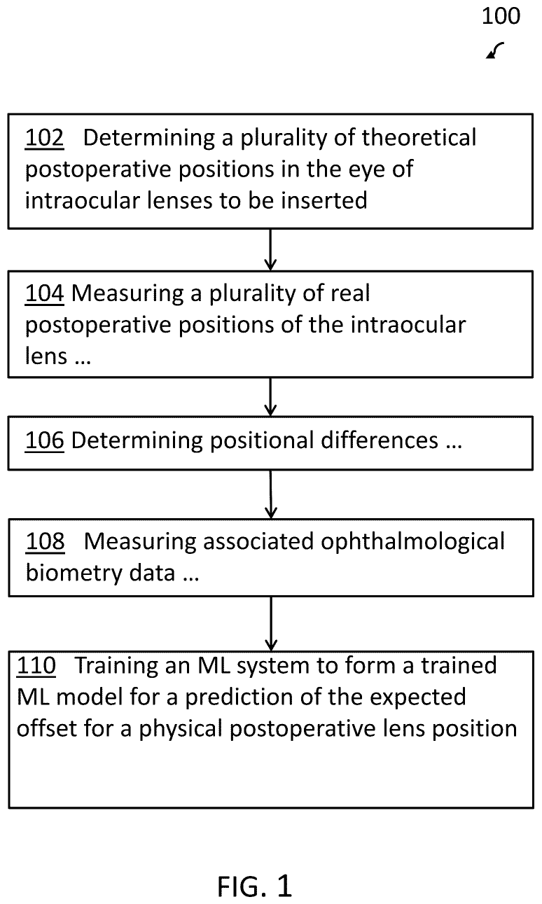

FIG. 1 illustrates a flowchart-like representation of a preferred exemplary embodiment of the computer-implemented method 100 for training a machine learning system to determine an expected offset for a physical postoperative lens position (i.e., PLP=postoperative anterior chamber depth) of an intraocular lens to be inserted. The method includes determining 102—i.e., collecting, registering, storing, evaluating, etc. —a plurality of theoretical postoperative positions in the eye of intraocular lenses to be inserted, with the intraocular lenses to be inserted belonging to different types of intraocular lenses (this also comprises the same types of IOL with different refractive powers). In this case, the determination for the plurality of theoretical postoperative positions includes a respective use of a relation—e.g., SKR/T*—and a respective lens-specific constant—in particular the type-dependent A-constant.

The method 100 furthermore comprises measuring 104 a plurality of real postoperative positions—in particular as ground truth data—of the intraocular lens using known measurement systems, with the real postoperative positions being assigned to respective associated theoretical postoperative positions and forming a tuple in each case.

The method 100 furthermore includes determining 106—e.g., calculating—positional differences between theoretical postoperative positions and real postoperative positions which are respectively associated with one another and measuring 108 associated ophthalmological biometry data for each tuple.

Finally, the method 100 includes training 110 a machine learning system—in a manner analogous to the aforementioned correction system—to form a trained machine learning model for a prediction of the expected offset for a physical postoperative lens position for a specific type of intraocular lens to be inserted, with the ophthalmological patient data for each tuple and associated positional differences being used as input data for training the machine learning system.

Large parts of the aforementioned advantages of the concept according to the disclosure can be achieved herewith. Continuing the aforementioned advantageous concept, a specific example is now presented hereinafter.

On the basis of the above-discussed advantageous theoretical background of SRK/T—and comparable formulas—it is possible to associate the mean chamber thickness (ACD=anterior chamber depth) after the operation, which corresponds to the real IOL position, with constants such as the A-constant, for example. In this context, the SRK/T-R relation can be specified as:

ACD constant=0.62467*A−constant−68.747,

(cf.] Retzlaff, John A. Sanders, Donald R. Kraff, Manus C., Development of the SRK/T intraocular lens implant power calculation formula, Journal of Cataract and Refractive Surgery, 16 (3), 333-340, 1990).

Hence the offset APLP can straight away be directly associated with the offset

ΔACD constant=0.62467*ΔA-constant.

Thus, a prediction system—i.e., a machine learning model for an underlying learning system—for predicting the postoperative, real IOL position is generated, which is to say trained, on this basis. This is based on two parts: the correction system which was trained by means of the training data and which predicts the difference between the measured, postoperative IOL position and the ACD constant as output data, and the A-constant as follows:

PLP=0.62467*A-constant−68.747+correction value

Such a prediction system now firstly allows the PLP to be predicted for a novel lens for which no postoperative biometric data are available. By way of example, this is implemented by inserting the appropriate A-constant or by way of a removal of the distribution offset for two correction systems which were trained for two different lens types if postoperative information is available for both lens types.

Advantageously, the proposed method consequently describes a general approach for compensating domain shifts between data regarding different IOL types, resulting in an enablement of an effective training for a machine learning system on the basis of data from different types of intraocular lenses within the same training data record.

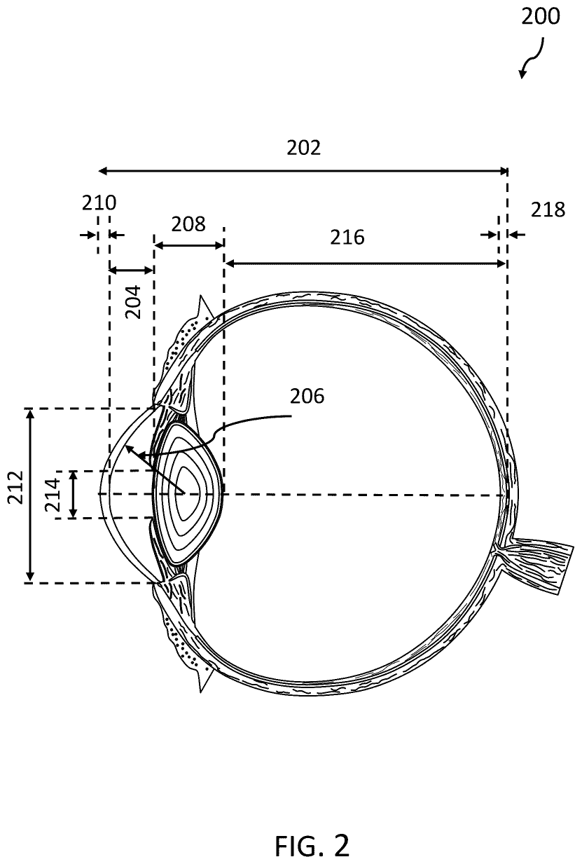

FIG. 2 depicts an eye 200 with different biometric parameters of the eye. In particular, the following parameters are displayed: axial length 202 (AL), anterior chamber depth 204 (ACD), keratometry value 206 (K, radius), refractive power of the lens, lens thickness 208 (LT), central cornea thickness 210 (CCT), white-to-white distance 212 (WTW), pupil size 214 (PS), posterior chamber depth 216 (PCD), retina thickness 218 (RT).

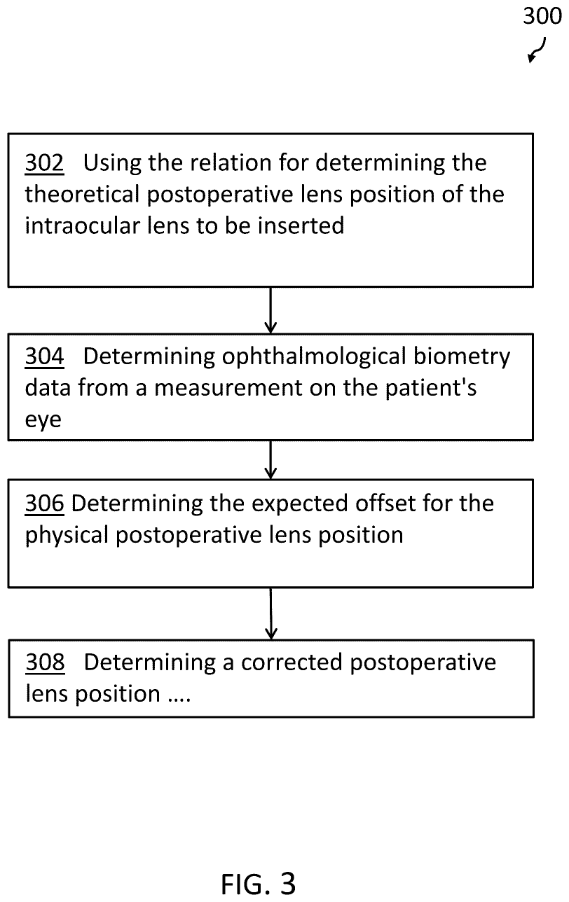

FIG. 3 shows a flowchart-like representation of an exemplary embodiment of a method 300 for determining a corrected postoperative lens position proceeding from the method according to FIG. 1, which is to say using the machine learning system trained by means of the above-described method. In this case, this method 300 includes using 302 the relation for determining the theoretical postoperative lens position of the intraocular lens to be inserted, and determining 304 ophthalmological biometry data from a measurement on the patient's eye—especially prior to the operation. In this case, determining the ophthalmological biometry data can be implemented in different ways, for example by a direct measurement, an evaluation of measurements, a retrieval from a memory, etc.

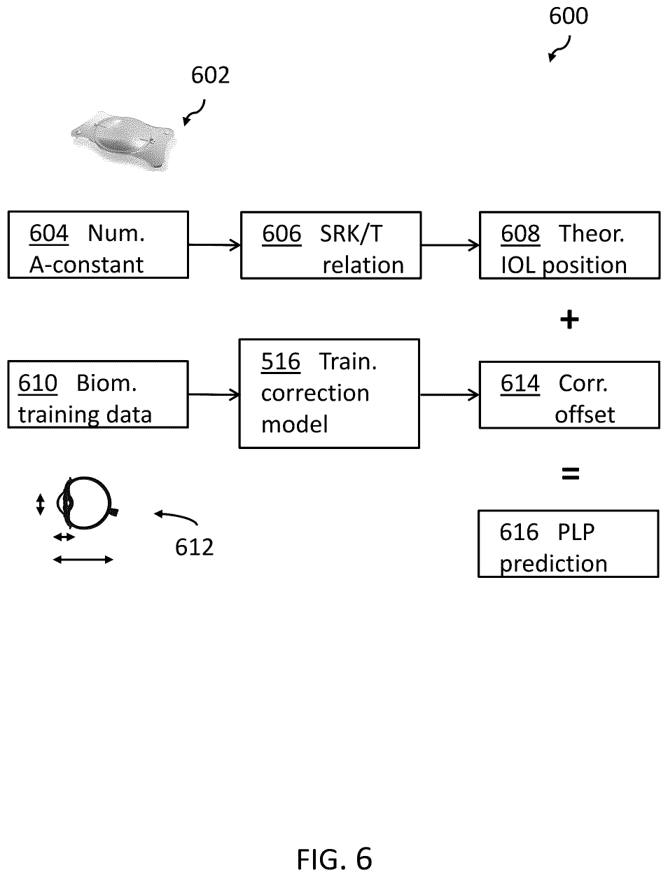

The method 300 also includes: determining 306, by means of the trained machine learning system, the expected offset for the physical postoperative lens position for an intraocular lens to be inserted, with the determined ophthalmological biometry data being used as input data, and determining 308 a corrected postoperative lens position by adding the theoretical postoperative lens position of the intraocular lens to be inserted and the determined expected offset for the physical postoperative lens position. This is vividly presented again hereinafter by FIG. 6.

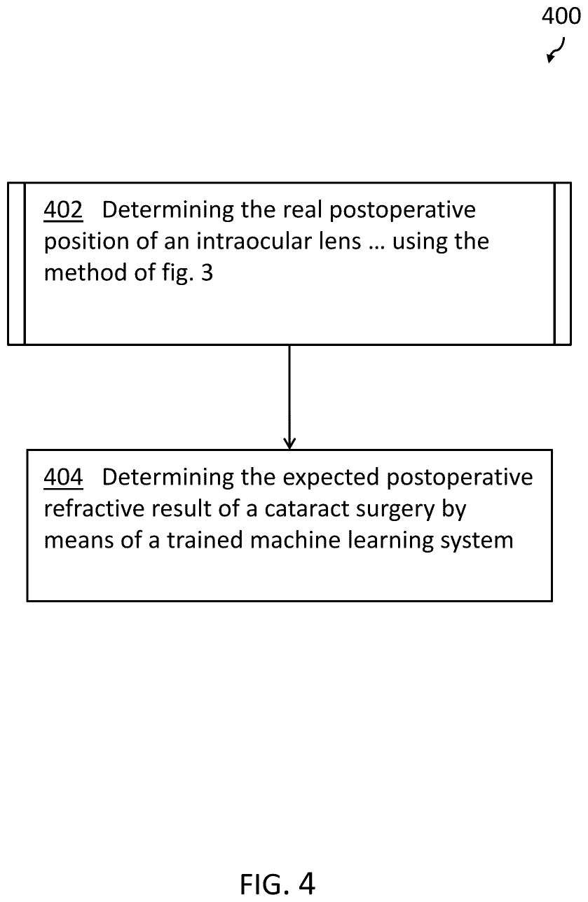

FIG. 4 shows a flowchart-like representation of an exemplary embodiment 400 for determining an expected postoperative refractive result of a cataract operation, for example. To this end, the above-described method 300 (cf. FIG. 3) for determining 402 the real postoperative position of an intraocular lens is initially resorted to.

This step is followed by the actual determination 404 of the expected postoperative refractive result of a cataract surgery by means of the trained machine learning system. In this case, a real refractive power value of the intraocular lens to be inserted and the determined postoperative real position of an intraocular lens are used as input data for a corresponding machine learning system.

This corresponding learning system was trained in advance with tuples of postoperative lens position values, real refractive power values of corresponding intraocular lenses, and associated postoperative refractive results as ground truth data, in order to form a corresponding learning model for predicting the postoperative refractive result of the intraocular lens to be inserted.

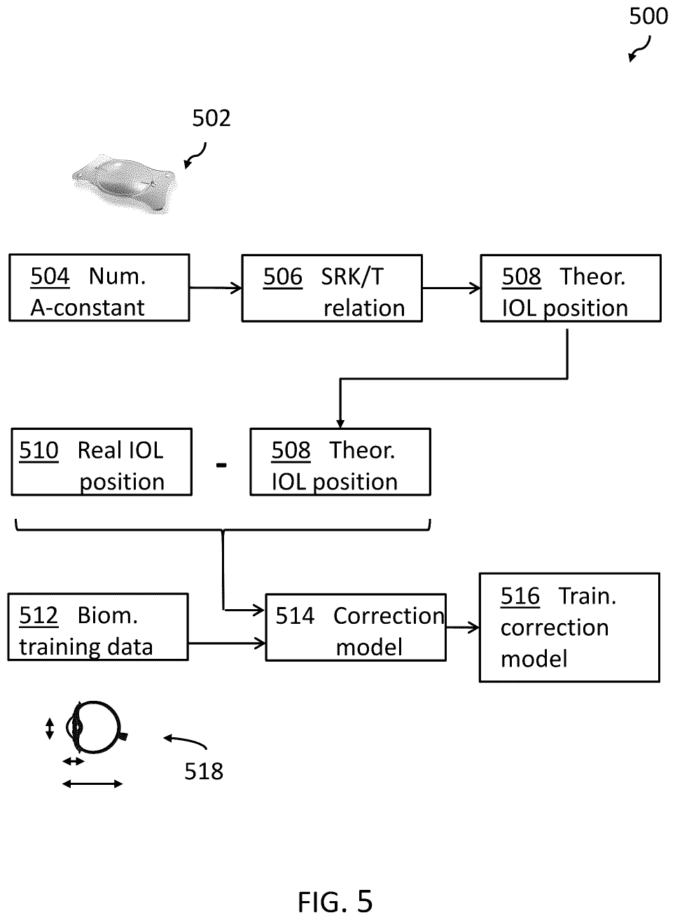

FIG. 5 takes up the training method again. Consequently, FIG. 5 illustrates a schematic representation for training a machine learning system in accordance with an exemplary embodiment of the presented concept. For a certain type of intraocular lens 502, the numerical A-constant 504 is used as an input value for the SRK/T relation 506, in order to calculate a theoretical IOL position 508. Then, the theoretical IOL position 508 is subtracted from the real, known IOL position 510 The result and the biometric training data (geometry data) 512 are used as input values for the correction model 514, and hence a trained correction model 516 is produced in the form of a machine learning system.

FIG. 6 again takes up the prediction phase for the postoperative position of the IOL to be inserted. Consequently, FIG. 6 illustrates a schematic representation 600 for a PLP prediction on the basis of a machine learning system which was trained by means of the training according to FIG. 5.

Proceeding from an intraocular lens 602 actually to be inserted, the numerical A-constant 604 thereof is used to determine the theoretical IOL position 608 by means of the SRK/T relation 606. On the other side, the biometric data 612 (cf. also FIG. 5: 518) in the form of the biometric training data 610 for the trained correction model 516 are available. The latter predicts the correction offset 614 in relation to the lens position. Adding the theoretical IOL position 608 and the correction offset 614 thus ultimately yields a PLP prediction 616.

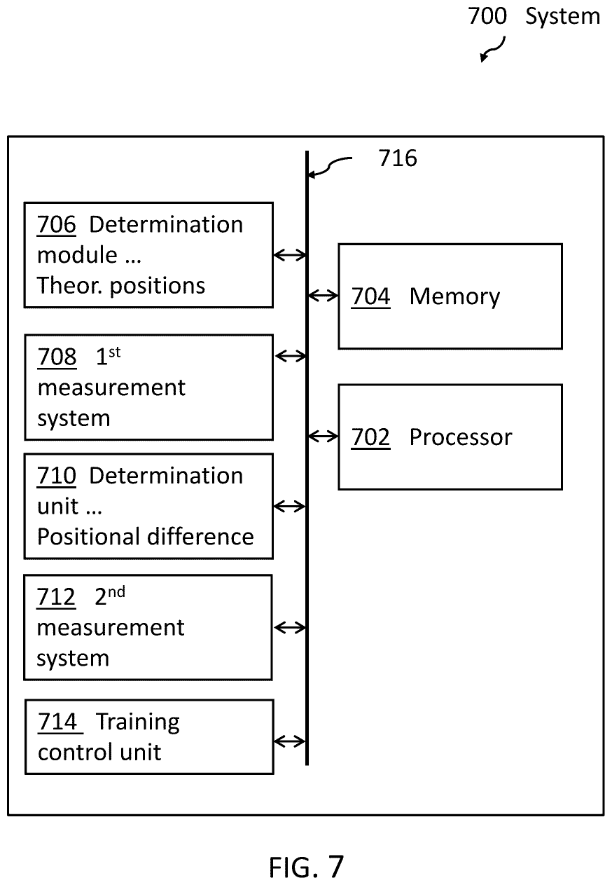

FIG. 7 shows a schematic representation of a training system 700 for training a machine learning system to determine an expected offset for a physical postoperative lens position of an intraocular lens to be inserted. In this case, the training system 700 comprises a processor 702 and a memory 704 which is operatively connected to the processor 702 and which stores program code elements which, when executed, are active for an activation and cooperation of the following units:
(i) a determination module 706 for determining a plurality of theoretical postoperative positions in the eye of intraocular lenses to be inserted, the intraocular lenses to be inserted belonging to different types of intraocular lenses,
the determination including a respective use of a relation and a respective lens-specific constant for the plurality of the theoretical postoperative positions,
(ii) a first measurement system 708 for measuring a plurality of real postoperative positions of the intraocular lens, with the real postoperative positions being assigned to respective associated theoretical postoperative positions and forming a tuple in each case,
(iii) a determination unit 710 for determining positional differences between theoretical postoperative positions and real postoperative positions which are associated with one another in each case,
(iv) a second measurement system 712 for measuring associated ophthalmological biometry data for each tuple, and
(v) a training control unit 714 for training a machine learning system to form a trained machine learning model for a prediction of the expected offset for a physical postoperative lens position for a specific type of intraocular lens to be inserted, with the ophthalmological patient data for each tuple and associated positional differences being used as input data for training the machine learning system.

Express reference is made to the fact that the aforementioned modules and units—in particular the processor 702, the memory 704, the determination module 706, the 1st measurement system 708, the determination system 710, the 2nd measurement system 712 and the training control unit 714—can be connected using electrical signal lines or via a system-internal bus system 716 for the purpose of exchanging signals or data.

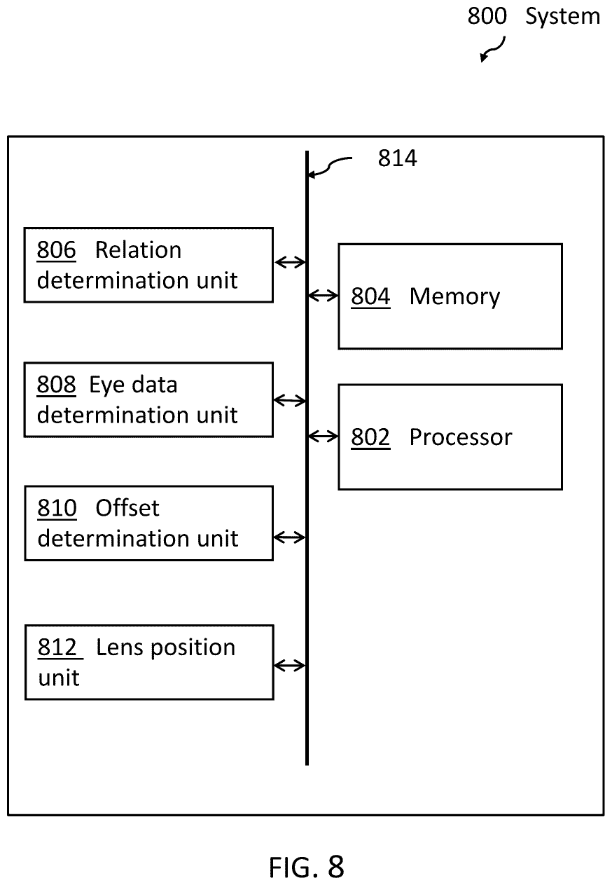

FIG. 8 shows a schematic representation for a prediction system 800 for determining a postoperative real position of an intraocular lens using the machine learning system that was trained by means of the training system. In this case, the prediction system comprises a processor 802 and a memory 804 which is operatively connected to the processor 802 and which stores program code elements which, when executed, are active for a cooperation of the following units:
(i) a relation determination unit 806 for using the relation for determining the theoretical postoperative lens position of the intraocular lens to be inserted,
(ii) an eye data determination unit 808 for determining ophthalmological biometry data from a measurement on the patient's eye,
(iii) an offset determination unit 810 for determining, by means of the trained machine learning system, the expected offset for the physical postoperative lens position for an intraocular lens to be inserted, with the determined ophthalmological biometry data being used as input data, and
(iv) a lens position unit for determining a corrected postoperative lens position by adding the theoretical postoperative lens position of the intraocular lens to be inserted and the determined expected offset for the physical postoperative lens position.

Express reference is also made here to the fact that the aforementioned modules and units—in particular the processor 802, the memory 804, the relation determination unit 806, the eye data determination unit (measurement system for detecting biometric data) 808, the offset determination unit 810 and the lens position unit 812—can be connected using electrical signal lines or via a system-internal bus system 814 for the purpose of exchanging signals or data.

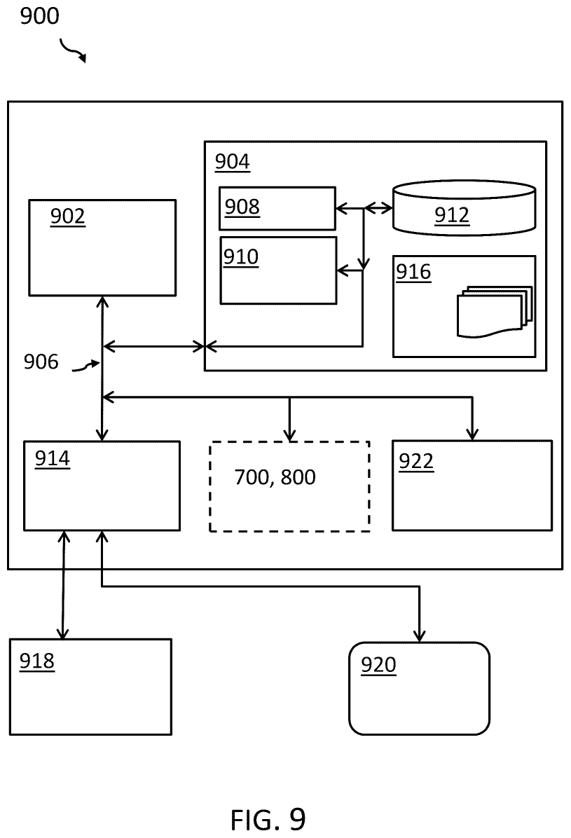

FIG. 9 describes a computer system 900 which can be used in the context of the systems already described herein. The computer system 900 has a plurality of general-purpose functions. The computer system may in this case be a tablet computer, a laptop/notebook computer, some other portable or mobile electronic device, a microprocessor system, a microprocessor-based system, a smartphone, a computer system with specially configured special functions, or else a constituent part of a microscope system. The computer system 900 may be configured so as to execute computer system-executable instructions—such as for example program modules—that may be executed in order to implement functions of the concepts proposed here. For this purpose, the program modules can comprise routines, programs, objects, components, logic, data structures etc. in order to implement particular tasks or particular abstract data types.

The components of the computer system may comprise the following: one or more processors or processing units 902, a storage system 904 and a bus system 906 that connects various system components, including the storage system 904, to the processor 902. The computer system 900 typically comprises a plurality of volatile or non-volatile storage media accessible by the computer system 900. The storage system 904 can store the data and/or instructions (commands) of the storage media in volatile form—such as for example in a RAM (random access memory) 908—in order to be executed by the processor 902. These data and instructions realize one or more functions and/or steps of the concept presented here. Further components of the storage system 904 may be a permanent memory (ROM) 910 and a long-term memory 912, in which the program modules and data (reference sign 916) and also workflows may be stored.

The computer system has a number of dedicated devices (keyboard 918, mouse/pointing device [not illustrated], screen 920, etc.) for communication purposes. These dedicated devices can also be combined in a touch-sensitive display. An I/O controller 914, provided separately, ensures a frictionless exchange of data with external devices. A network adapter 922 is available for communication via a local or global network (LAN, WAN, for example via the Internet). The network adapter can be accessed by other components of the computer system 900 via the bus system 906. It is understood in this case, although it is not illustrated, that other devices can also be connected to the computer system 900.

Additionally, at least parts of the training system 700 for training a machine learning system to determine an expected offset for a physical postoperative lens position of an intraocular lens to be inserted (cf. FIG. 7) can be connected to the bus system 906, just like the system according to FIG. 8. In the process, the system according to FIG. 7 and/or according to FIG. 8 and the computer system 900 can, possibly in part, jointly use bus systems, memories and/or processors.

The description of the various exemplary embodiments of the present disclosure has been given for the purpose of improved understanding, but does not serve to directly restrict the inventive concept to these exemplary embodiments. A person skilled in the art will themselves develop further modifications and variations. The terminology used here has been selected so as to best describe the basic principles of the exemplary embodiments and to make them easily accessible to a person skilled in the art.

The principle proposed here may be embodied both as a system, as a method, combinations thereof and/or as a computer program product. The computer program product can in this case comprise one (or more) computer-readable storage medium/media comprising computer-readable program instructions in order to cause a processor or a control system to implement various aspects of the present disclosure.

Electronic, magnetic, optical, electromagnetic or infrared media or semiconductor systems are used as forwarding medium; for example SSDs (solid state devices/drives as solid state memory), RAM (random access memory) and/or ROM (read-only memory), EEPROM (electrically erasable ROM) or any combination thereof. Suitable forwarding media also include propagating electromagnetic waves, electromagnetic waves in waveguides or other transmission media (for example light pulses in optical cables) or electrical signals transmitted in wires.

The computer-readable storage medium may be an embodying device that retains or stores instructions for use by an instruction execution device. The computer-readable program instructions that are described here can also be downloaded onto a corresponding computer system, for example as a (smartphone) app from a service provider via a cable-based connection or a mobile radio network.

The computer-readable program instructions for executing operations of the disclosure described here may be machine-dependent or machine-independent instructions, microcode, firmware, status-defining data or any source code or object code that is written for example in C++, Java or the like or in conventional procedural programming languages such as for example the programming language “C” or similar programming languages. The computer-readable program instructions may be executed in full by a computer system. In some exemplary embodiments, there may also be electronic circuits, such as, for example, programmable logic circuits, field-programmable gate arrays (FPGAs) or programmable logic arrays (PLAs), which execute the computer-readable program instructions by using status information of the computer-readable program instructions in order to configure or to individualize the electronic circuits according to aspects of the present disclosure.

The disclosure proposed here is furthermore illustrated with reference to flowcharts and/or block diagrams of methods, apparatuses (systems) and computer program products according to exemplary embodiments of the disclosure. It should be pointed out that practically any block of the flowcharts and/or block diagrams can be embodied as computer-readable program instructions.

The computer-readable program instructions may be made available to a general-purpose computer, a special computer or a data processing system able to be programmed in another way in order to create a machine such that the instructions that are executed by the processor or the computer or other programmable data processing devices generate means for implementing the functions or procedures that are illustrated in the flowchart and/or block diagrams. These computer-readable program instructions can correspondingly also be stored on a computer-readable storage medium.

In this sense, any block in the illustrated flowchart or the block diagrams may represent a module, a segment or portions of instructions that represent a plurality of executable instructions for implementing the specific logic function. In some exemplary embodiments, the functions represented in the individual blocks can be implemented in a different order—optionally also in parallel.

The illustrated structures, materials, sequences, and equivalents of all of the means and/or steps with associated functions in the claims below are intended to apply all of the structures, materials or sequences as expressed by the claims.

What is claimed is:

1. A computer-implemented method for training a machine learning system to determine an expected offset for a physical postoperative lens position of an intraocular lens to be inserted, the method comprising:
determining a plurality of theoretical postoperative positions in the eye of intraocular lenses to be inserted, the intraocular lenses to be inserted belonging to different types of intraocular lenses, the determination including a respective use of a relation and a respective lens-specific constant for the plurality of the theoretical postoperative positions;
measuring a plurality of real postoperative positions of the intraocular lens, with the real postoperative positions being assigned to respective associated theoretical postoperative positions and forming a tuple in each case;
determining positional differences between theoretical postoperative positions and real postoperative positions which are respectively associated with one another;
measuring associated ophthalmological biometry data for each tuple; and
training a machine learning system to form a trained machine learning model for a prediction of the expected offset for a physical postoperative lens position for a specific type of intraocular lens to be inserted, with the ophthalmological biometry data for each tuple and associated positional differences being used as input data for training the machine learning system.

2. The method of claim 1, wherein the relation is based on an SRK/T relation.

3. The method of claim 1, wherein the ophthalmological biometry data include at least one measurement value of an eye selected from the group consisting of a current axial length (AL), an anterior chamber depth (ACD), and a lens thickness.

4. The method of claim 1, wherein the machine learning system includes a linear regression model.

5. The method of claim 1, wherein the prediction of the expected offset for the physical postoperative lens position for the specific type of intraocular lens to be inserted is independent of a refractive power of the intraocular lens to be inserted.

6. The method of claim 1, wherein the lens-specific constant is the A-constant.

7. A method for determining a real postoperative position of an intraocular lens using the machine learning system which was trained by means of the method according claim 1, the method comprising:
using the relation for determining the theoretical postoperative lens position of the intraocular lens to be inserted;
determining ophthalmological biometry data from a measurement on the patient's eye;
determining, by means of the trained machine learning system, the expected offset for the physical postoperative lens position for an intraocular lens to be inserted, with the determined ophthalmological biometry data being used as input data; and
determining a corrected postoperative lens position by adding the theoretical postoperative lens position of the intraocular lens to be inserted and the determined expected offset for the physical postoperative lens position.

8. A method for determining a postoperative refractive result, the method including:
determining the real postoperative position of an intraocular lens using the method of claim 7;
determining the expected postoperative refractive result of a cataract surgery by means of a trained machine learning system, with a real refractive power value of the intraocular lens to be inserted and the determined real postoperative position of an intraocular lens being used as input data;
the machine learning system having been trained with tuples of postoperative lens position values, real refractive power values of corresponding intraocular lenses, and associated postoperative refractive results as ground truth data, in order to form a corresponding learning model for predicting the postoperative refractive result of the intraocular lens to be inserted.

9. A training system for training a machine learning system to determine an expected offset for a physical postoperative lens position of an intraocular lens to be inserted, the training system comprising:
a processor and a memory which is operatively coupled to the processor and which stores program code elements, which, when executed, are active for a cooperation of the following units:
a determination module for determining a plurality of theoretical postoperative positions in the eye of intraocular lenses to be inserted, the intraocular lenses to be inserted belonging to different types of intraocular lenses, the determination including a respective use of a relation and a respective lens-specific constant for the plurality of the theoretical postoperative positions;
a first measurement system for measuring a plurality of real postoperative positions of the intraocular lens, with the real postoperative positions being assigned to respective associated theoretical postoperative positions and forming a tuple in each case;
a determination unit for determining positional differences between theoretical postoperative positions and real postoperative positions which are associated with one another in each case;
a second measurement system for measuring associated ophthalmological biometry data for each tuple; and
a training control unit for training a machine learning system to form a trained machine learning model for a prediction of the expected offset for a physical postoperative lens position for a specific type of intraocular lens to be inserted, with the ophthalmological patient data for each tuple and associated positional differences being used as input data for training the machine learning system.

10. The system of claim 9, wherein the relation is based on an SRK/T relation.

11. The system of claim 9, wherein the ophthalmological biometry data include at least one measurement value of an eye selected from the group consisting of a current axial length (AL), an anterior chamber depth (ACD), and a lens thickness.

12. The system of claim 9, wherein the machine learning system includes a linear regression model.

13. The system of claim 9, wherein the prediction of the expected offset for the physical postoperative lens position for the specific type of intraocular lens to be inserted is independent of a refractive power of the intraocular lens to be inserted.

14. The system of claim 9, wherein the lens-specific constant is the A-constant.

15. A prediction system for determining a real postoperative position of an intraocular lens using the machine learning system which was trained by means of the training system of claim 9, the prediction system including:
a processor and a memory which is operatively coupled to the processor and which stores program code elements, which, when executed, are active for a cooperation of the following units:
a relation determination unit for using the relation for determining the theoretical postoperative lens position of the intraocular lens to be inserted;
an eye data determination unit for determining ophthalmological biometry data from a measurement on the patient's eye;
an offset determination unit for determining, by means of the trained machine learning system, the expected offset for the physical postoperative lens position for an intraocular lens to be inserted, with the determined ophthalmological biometry data being used as input data; and
a lens position unit for determining a corrected postoperative lens position by adding the theoretical postoperative lens position of the intraocular lens to be inserted and the determined expected offset for the physical postoperative lens position.

16. A computer program product for training a machine learning system for a determination of an expected offset for a physical postoperative lens position of an intraocular lens to be inserted, the computer program product having a computer-readable storage medium having program instructions stored thereon, the program instructions being executable by one or more computers or control units and prompting the one or more computers or control units to carry out the method according to claim 1.
